(19) United States
(12) Patent Application Publication
(10) Pub. No.: US 2007/0208414 A1
(43) Pub. Date: Sep. 6, 2007

(54) Tapered strength rings on a bifurcated stent petal

(76) Inventors: Shawn Sorenson, Maple Grove, MN (US); Richard C. Tooley, Crystal, MN (US); Michael P. Meyer, Richfield, MN (US); Daniel Gregorich, St. Louis Park, MN (US)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 11/368,846

(22) Filed: Mar. 6, 2006

(51) Int. Cl. A61F 2/94

(52) U.S. Cl. 623/1.16; 623/1.35

(57) ABSTRACT

A stent assembly comprises a second stent body and a substantially tubular first stent body defining a first lumen and containing a side branch opening. In an undeployed state, the second stent body is at least partially comprised of rings having peaks and spans between these peaks. In a deployed state, the rings of the second stent body define a second lumen opening in fluid communication with the first lumen. The second lumen is capable of having tapering strength and geometry and can extend and form an oblique angle relative to the longitudinal axis of the first stent body.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use. Some embodiments are directed to delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be implanted to prevent restenosis following angioplasty in the vascular system. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

This invention contemplates a number of embodiments where any one, any combination of some, or all of the embodiments can be incorporated into a stent and/or a stent delivery system and/or a method of use.

At least one embodiment is directed to a stent assembly having an unexpanded state and an expanded state comprising: a substantially tubular, first stent body defining a first lumen with a proximal end and a distal end. The first lumen is positioned within a circumferential plane, has a first longitudinal axis extending through the circumferential plane and has a side opening along the circumferential plane. In addition, the system comprises a second stent body which in the unexpanded state is connected to the first stent body, positioned adjacent to the side opening substantially along the circumferential plane of the first stent body, and itself comprises a plurality of interconnected rings, each ring having peaks and spans between the peaks. At least one of the rings is an inner ring and at least one is an outer ring, and at least one ring is stronger than at least one other ring. In the expanded state, the interconnected rings define a second fluid lumen having a second longitudinal axis extending therethrough at an oblique angle to the first longitudinal axis. The second lumen has an ostial and an outermost end, and is connected to and in fluid communication with the first lumen. The ostial ring can be positioned adjacent to the side opening and can define the ostial end of the lumen. The outermost ring can be positioned at the opposite end of the second lumen to define the outer end.

At least one embodiment is directed to a bifurcated stent assembly where the bifurcation at least partially comprises a plurality of rings with peaks and spans in which the ostial ring is the strongest ring.

At least one embodiment is directed to a bifurcated stent assembly where the bifurcation at least partially comprises a plurality of rings with peaks and spans in which the outermost ring is the strongest ring.

At least one embodiment is directed to a bifurcated stent assembly where the bifurcation at least partially comprises a plurality of rings with peaks and spans in which the ostial ring is the weakest ring.

At least one embodiment is directed to a bifurcated stent assembly where the bifurcation at least partially comprises a plurality of rings with peaks and spans in which the outermost ring is the weakest ring.

At least one embodiment is directed to a bifurcated stent assembly where the bifurcation at least partially comprises a plurality of rings with peaks and spans in which the rings progressively weaken from the inner ring to the outer ring.

At least one embodiment is directed to a bifurcated stent assembly where the bifurcation at least partially comprises a plurality of rings with peaks and spans in which the rings progressively strengthen from the inner ring to the outer ring.

At least one embodiment is directed to a bifurcated stent assembly where the bifurcation at least partially comprises a plurality of rings with peaks and spans in which in the unexpanded state, the rings fit concentrically within each other.

At least one embodiment is directed to a bifurcated stent assembly where the bifurcation at least partially comprises a plurality of rings with peaks and spans in which each ring has an undeployed diameter and surrounding a center point and where at least two rings have unequal distances between their center points and their peaks.

At least one embodiment is directed to a bifurcated stent assembly where the bifurcation at least partially comprises a plurality of rings with peaks and spans in which the product of the number of peaks of one ring multiplied by the length of the span of that ring is equal to the product of the number of peaks of a second ring multiplied by the length of the span of that ring.

At least one embodiment is directed to a bifurcated stent assembly where the bifurcation at least partially comprises a plurality of rings with peaks and spans in which at least two rings have equal numbers of peaks.

At least one embodiment is directed to a bifurcated stent assembly where the bifurcation at least partially comprises a plurality of rings with peaks and spans in which at least two rings have unequal numbers of peaks.

At least one embodiment is directed to a bifurcated stent assembly where the bifurcation at least partially comprises a plurality of rings with peaks and spans in which at least one ring has 8 peaks and another has 16 peaks.

At least one embodiment is directed to a bifurcated stent assembly where the bifurcation at least partially comprises a plurality of rings with peaks and spans in which at least one ring has 8 peaks and another has 24 peaks.

At least one embodiment is directed to a bifurcated stent assembly where the bifurcation at least partially comprises a plurality of rings with peaks and spans in which at least two rings in the unexpanded state have different numbers of peaks and in the expanded state form substantially equal diameters.

At least one embodiment is directed to a bifurcated stent assembly where the bifurcation at least partially comprises a plurality of rings with peaks and spans in which at least two rings in the expanded state form substantially tapered diameters.

At least one embodiment is directed to a bifurcated stent assembly where the bifurcation at least partially comprises a plurality of rings with peaks in which in the unexpanded state at least two rings have 16 peaks.

At least one embodiment is directed to a bifurcated stent assembly where the bifurcation at least partially comprises a plurality of rings with peaks in which in the unexpanded state at least two rings have 16 peaks and another has 24 peaks.

At least one embodiment is directed to a bifurcated stent assembly where the bifurcation at least partially comprises a plurality of rings with peaks in which in the unexpanded state, the product of the number of peaks of one ring multiplied by the length of the span of that ring is greater than the product of number of peaks of a second ring multiplied by the length of the span of that ring and form a tapered second lumen in the expanded state.

At least one embodiment is directed to a bifurcated stent assembly where the bifurcation at least partially comprises a plurality of rings with peaks in which in the rings have equal numbers of peaks.

At least one embodiment is directed to a bifurcated stent assembly where the bifurcation at least partially comprises a plurality of rings in which the rings have unequal numbers of peaks.

In at least one embodiment, the self expansion mechanism includes biased members of the side branch assembly which in the unexpanded state are restrained by blocking struts of the main stent body and in the expanded state are released when restraining struts of the main stent body are withdrawn.

In at least one embodiment the stent has a plurality of side branch openings each with an area.

In at least one embodiment the stent has a plurality of side branch openings each with an area and the area of at least one side branch opening is greater than or smaller than that of each of the remaining openings.

In at least one embodiment the stent has a plurality of side branch openings and the first side branch opening and the second side branch opening are coaxially positioned relative to one another.

In at least one embodiment, the first stent body has an end-to-end length and the second stent body has an end-to-end length, and the end-to-end length of the second body is shorter than the end-to-end length of the first stent body.

In at least one embodiment, when in the undeployed state, no first stent body members are positioned across the second side branch opening.

In at least one embodiment, the self expansion mechanism is a constrained by a sheath surrounding the stent until after expansion, which when withdrawn, allows the side branch assembly to self expand.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a view of a side branch assembly of a bifurcated stent in which the side branch assembly comprises concentric rings of decreasing strength as the side branch extends into the body vessel branch.

FIG. 2 is a view of a side branch assembly of a bifurcated stent in which the side branch assembly comprises concentric rings of increasing strength as the side branch extends into the body vessel branch.

FIG. 3 is a view of two concentric rings of a side branch assembly of a bifurcated stent in which the ring of the two closer to the ostium has 8 peaks and the ring further extended into the vessel side branch has 16 peaks.

FIG. 4 is a view of two concentric rings of a side branch assembly of a bifurcated stent in which the ring of the two closer to the ostium has 24 peaks and the ring further extended into the vessel side branch has 8 peaks.

FIG. 5 is a view of two concentric rings of a side branch assembly of a bifurcated stent in which the ring of the two closer to the ostium has 8 peaks and the ring further extended into the vessel side branch has 8 peaks.

FIG. 6 is a view two concentric rings of a side branch assembly of a bifurcated stent in which in there are two rings with 8 peaks

FIG. 7 is a view of three concentric rings of a side branch assembly of a bifurcated stent in which all three rings have 8 peaks.

FIG. 8 is a view of three concentric rings of a side branch assembly of a bifurcated stent in which the ring of the three closest to the ostium has 32 peaks, the next closest has 8 peaks, and the ring further extended into the vessel side branch has 16 peaks.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

Depicted in the figures are various aspects of the invention. Elements depicted in one figure may be combined with, and/or substituted for, elements depicted in another figure as desired.

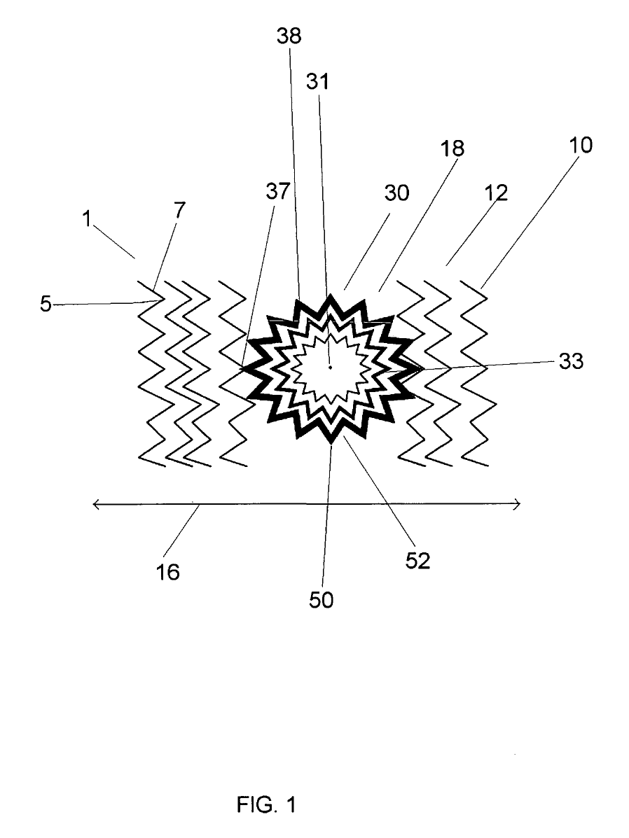

Referring now to FIG. 1 there is shown a portion of an unexpanded bifurcated stent 1 extending along a first longitudinal axis 16 having a first stent body 10 with a side opening 18. Although this illustration shows a first stent body 1 having a plurality of struts 5 forming columns 7, it encompasses all stent structures currently known in the art. In the expanded state, the first stent body will define a first fluid lumen 14 having a proximal end and a distal end. This first stent body 10 can be constructed at least partially out of a number of materials including but not limited to polymers, stainless steel, platinum, gold, cobalt, chromium, niobium etc. It can also be constructed out of one or more combinations and/or alloys of these materials.

Connected to the first stent body 1 there is a second stent body which is a side branch assembly 30 adjacent to the side opening 18. When in an expanded state, the side branch assembly 30 will define a second fluid lumen in fluid communication with the first fluid lumen 14. The region of the side branch assembly 30 is generally connected to the first stent body 10 and when in an expanded state will comprise the base of the second fluid lumen. This base of the second fluid lumen is referred to as the “ostium” 38. For purposes of this application and in particular when describing the drawings, when discussing a set of items, the item described as “ostial” is the item of the set closest to the ostium and the item described as “outermost” is the item of the set furthest away from the ostium.

The side branch assembly 30 comprises at least two rings 33 each positioned in a nested formation in the unexpanded state. For purposes of this application the term “nested” includes but is not limited to concentric, stacked, overlapping, and adjacent formations. As the stent 1 expands, at least one ring extends away from the ostium 38 into the body vessel branch forming a generally serial configuration with the other ring(s). The extended rings 33 together define at least a portion of a generally tubular secondary fluid lumen in fluid communication with the first fluid lumen 14. At least a portion of the secondary fluid lumen is bound between a ring closest to the ostium 38 and an outermost ring. Some or all of the rings can be connected to each other by ring-ring connectors or to the first stent body by ring-stent connectors. In one possible embodiment, each ring is connected to an adjacent ring by a ring-ring connector and only the ostial ring is connected to the first stent body by a ring-stent connector.

At least one of the rings comprises a plurality of peaks 50 and spans 52 between the peaks 50. In FIG. 1, the span is represented by an angled strut, but the span can define any connecting member(s) located between the two peaks. The rings can have varying physical properties such as resistance to radial compression, resistance to expansion, longitudinal stiffness, different hardness's, and different modulus of elasticity with for example a ring with one array of physical properties located closest to the ostium one with another array further down the side branch. The differences in properties can be accomplished by using rings with different widths or thicknesses or by using stronger, denser, and more rigid materials. For purposes of this application the term “width” refers to area measured radially from the center point 31 of the rings and “thickness” refers to area measured along an axis generally parallel to the second fluid lumen formed when the stent is deployed. Locating the hardest, widest, thickest or densest ring at the ostium allows for easy initial opening of the side branch during deployment. Although FIG. 1 illustrates the side branch assembly with the rings decreasing in width progressively as they are positioned along the side branch, this invention contemplates possible embodiments in which the rings can have differing or equal thicknesses, hardness, size, density, radial strength, elasticity, or other physical properties and can be positioned in any sequence or order. At least some of the physical properties of the rings can be changed by modifying the thickness and material out of which the rings are constructed.

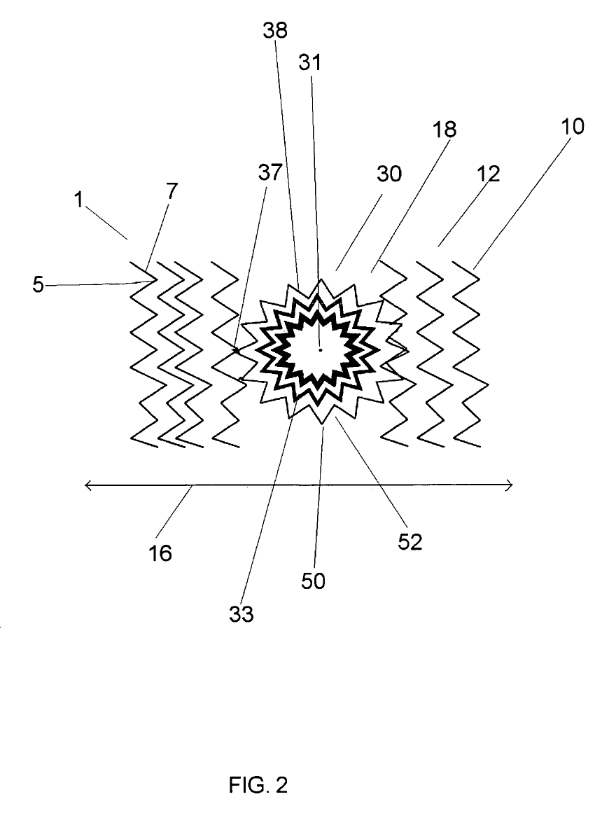

Referring now to FIG. 2 there is shown a side branch assembly design in which the ring at the ostium is the least wide of the rings and the outermost ring is the widest. This configuration can make drug delivery more efficacious, can allow for better vessel support, and can better hold the outermost ring in place. This invention also encompasses embodiments where the ring at the outermost end is the hardest, thickest, or densest of the rings and the rings progressively decrease in hardness, thickness, or density from the outermost end to the ostial end.

This invention also includes possible embodiments in which the rings of the secondary fluid lumen form equally sized diameters, varying sized diameters, a tapered diameter narrowest at the ostium, a tapered diameter narrowest at the outermost ring, a tapered diameter narrowest at the end of the secondary lumen farthest away from the ostium, or any combination of these configurations. In addition, expanded rings with different widths can form a secondary lumen with a generally tubular circumferential plane but which has a tapered interior because of the progressively changing ring thickness.

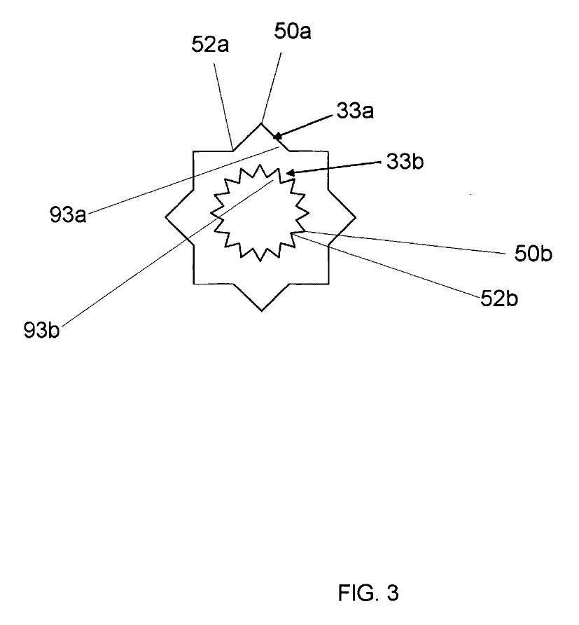

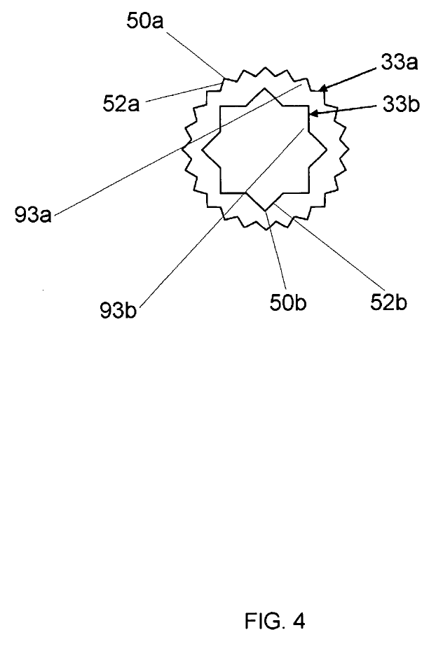

Referring now to FIGS. 3 and 4 there is shown one possible embodiment of this invention where the rings which define the secondary fluid lumen in the expanded state have uniform maximum expandable diameters and in the unexpanded state are concentrically positioned and are unequally sized. These figures illustrate a design for proportioning a set of rings based on the “equal diameter formula” defined as: n₁*L₁=n₂*L₂. In the equal diameter formula n₁ represents the number of peaks in the ostial ring, n₂ represents the number of peaks in the outermost ring, L₁ represents the relative length of the span in the ostial ring, and L₂ represents the relative length of the span in the outermost ring. FIG. 3 illustrates two rings 33a and 33b in which the outermost ring 33a has 16 peaks and the ostial ring 33b has 8 peaks. So long as the lengths of the inter-peak spans 93a and 93b in the two rings are proportioned according to the equal diameter formula they will have equal maximum expansion diameters. Of course in practice when expanded in a body vessel, the actual resulting diameters can possibly vary because of differing geometry at different positions along the body vessel branch.

Referring now to FIG. 4 there is shown another possible peaked ring configuration designed according to the equal diameter formula in which the ostial ring has 24 peaks and the outermost ring has 8 peaks.

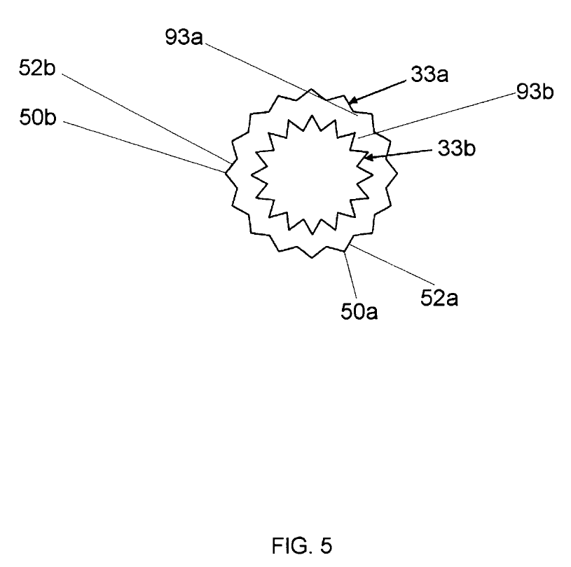

Referring now to FIG. 5 there is shown another possible peaked ring configuration designed according to the equal diameter formula in which the ostial ring has 16 peaks and the outermost ring has 16 peaks. Because both rings have equal numbers of peaks they also have equal span lengths.

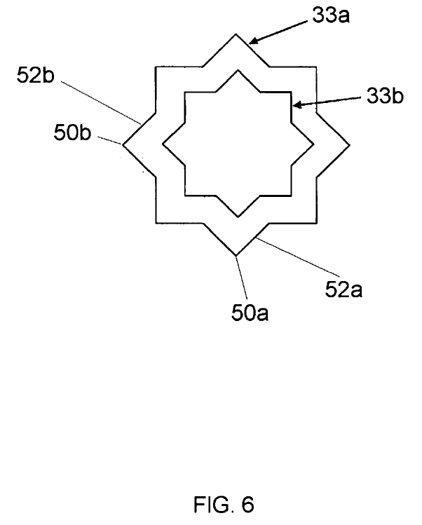

Referring now to FIGS. 6, 7, and 8 there are shown ring configurations designed to form a tapered secondary lumen in which the ostial end of the lumen is has the largest diameter. These designs make use of the “tapering diameter formula” defined as: n₁*L₁>n₂*L₂ or in designs with three rings: n₁*L₁>n₂*L₂>n₃*L₃. In the tapering diameter formula n₁ represents the number of peaks in the ostial ring, n₂ represents the number of peaks in the ring adjacent to the ostial ring and n₃ represents the represents the number of peaks in the outermost ring. Similarly, L₁ represents the relative length of the span in ostial ring, L₂ represents the relative length of the span in the ring adjacent to the ostial ring, and L₃ represents the relative length of the span in the outermost ring. In a tapered configuration the width of the secondary fluid lumen progressively increases or decrease along the length of the secondary lumen.

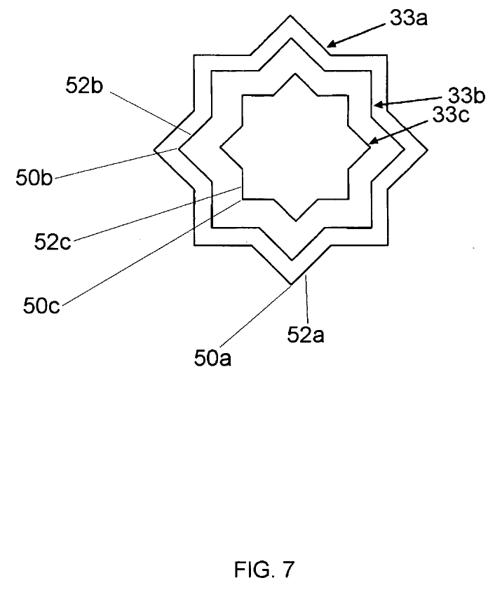

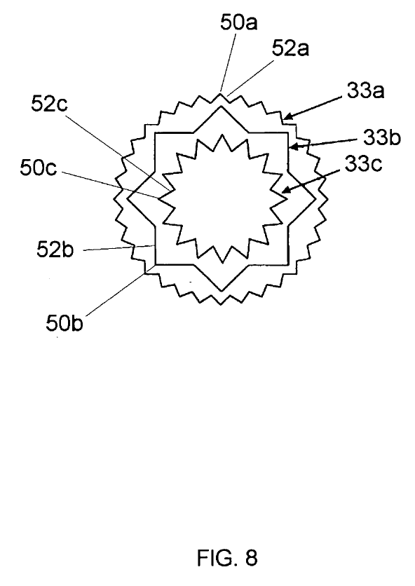

FIG. 6 illustrates tapering side branch rings designed according to the tapering diameter formula in which two rings have 8 peaks. FIG. 7 illustrates tapering side branch rings designed according to the tapering diameter formula in which three rings have 8 peaks. FIG. 8 illustrates tapering side branch rings designed according to the tapering diameter formula in the ostial ring has 32 peaks, the ring adjacent to the ostial ring has 8 peaks, and the outermost ring has 16 peaks.

The side branch assembly 30 when expanded into the second fluid lumen 34 has a second longitudinal axis 36 which forms an oblique angle 90 with the first longitudinal axis 16. For the purposes of this application, the term “oblique” refers to an angle of between 1 and 180 degrees and explicitly includes angles of about 90 degrees. Although FIGS. 1-8 show a single side branch opening and a singe side branch assembly, there can be multiple side branch openings and side branch assemblies. The sizes of the side branches can vary as well having larger, smaller or the same area, end-on-end length, or circumference in the extended or unextended states. Multiple side branch openings can be positioned anywhere along the length of the first stent body 1 and can be coaxially positioned relative to one another. This invention can be applied to both balloon expandable and self expanding stents. In addition, although FIGS. 1-8 show the side branch assembly 30 constructed in a petal type arrangement, this invention encompasses all forms of side branch assembles.

The inventive stents may be made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, niobium alloys, cobalt-chromium alloys including Elgiloy and Phynox, MP35N alloy and nickel-titanium alloys, for example, Nitinol.

The inventive stents may be made of shape memory materials such as superelastic Nitinol or spring steel, or may be made of materials which are plastically deformable. In the case of shape memory materials, the stent may be provided with a memorized shape and then deformed to a reduced diameter shape. The stent may restore itself to its memorized shape upon or after being heated to a transition temperature and having any restraints removed therefrom.

The inventive stents may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments the at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A bifurcated stent being expandable from an unexpanded state to an expanded state, wherein in the unexpanded state the stent has a diameter less than that of the diameter in the expanded state, the bifurcated stent comprising:
a substantially tubular first stent body defining a first circumferential plane, a first outer surface, a first lumen and having a proximal end, a distal end, a first longitudinal axis extending therethrough, and having a side opening along the first circumferential plane;
a second stent body positioned adjacent to the side opening and engaged to first stent body, the second stent body comprising a plurality of interconnected expandable rings at least two of the rings having progressively differing widths, in the unexpanded state the at least two rings being positioned substantially within the first circumferential plane, in the expanded state the at least two rings being positioned external to the first circumferential plane and defining a secondary circumferential plane, a secondary lumen, and having a secondary longitudinal axis extending therethrough, the secondary lumen being in fluid communication with the first lumen, the secondary longitudinal axis forming an oblique angle with the primary longitudinal axis.

2. The stent assembly of claim 1 having one ring being an ostial ring positioned closest to the junction of the first and second stent bodies and one ring being an outermost ring positioned farthest away from the junction of the first and second stent bodies wherein the ostial ring is positioned adjacent to the side opening and defines the ostium of the second lumen.

3. The stent assembly of claim 1 having one ring being an ostial ring positioned closest to the junction of the first and second stent bodies and one ring being an outermost ring positioned farthest away from the junction of the first and second stent bodies wherein the outermost ring is positioned at the opposite end of the second lumen of the ostium and the outermost ring defines the outer end of the second lumen.

4. The stent assembly of claim 3 in which the secondary lumen has a width and the width tapers between the ostial ring and the outermost ring.

5. The stent assembly of claim 3 in which the ostial ring is the widest ring.

6. The stent assembly of claim 3 in which the outermost ring is the widest ring.

7. The stent assembly of claim 1 having one ring being an ostial ring positioned closest to the junction of the first and second stent bodies and one ring being an outermost ring positioned farthest away from the junction of the first and second stent bodies wherein in the unexpanded state, the rings fit concentrically within each other.

8. The stent assembly of claim 1 in which in the unexpanded state, the rings further comprise a centerpoint, wherein at least two rings have unequal distances between their center points and their peaks.

9. The stent assembly of claim 8 in which in the expanded state, at least two of the rings have substantially equal diameters.

10. A bifurcated stent assembly having an expanded state and an unexpanded state, the assembly comprising:
a substantially tubular, first stent body defining a first lumen with a proximal end and a distal end, the first lumen positioned within a circumferential plane, having a first longitudinal axis extending therethrough and having a side opening along the circumferential plane;
and a second stent body;
the second stent body being connected to the first stent body, positioned adjacent to the side opening, and comprising a plurality of interconnected rings;
in the unexpanded state at least two of the rings being positioned substantially along the circumferential plane of the first stent body each of the at least two rings having a plurality of peaks and spans in between these peaks wherein the product of the number of peaks of one ring multiplied by the length of the span of that ring is equal to the product of number of peaks of a second ring multiplied by the length of the span of the second ring and;
in the expanded state, the interconnected rings of the second stent body are positioned farther away from each other than in the unexpanded state and define a second lumen and the second stent body is in fluid communication with the first fluid lumen.

11. The stent assembly of claim 10 wherein the at least two rings have equal numbers of peaks.

12. The stent assembly of claim 10 having one ring being an ostial ring positioned closest to the junction of the first and second stent bodies and one ring being an outermost ring positioned farthest away from the junction of the first and second stent bodies wherein the at least two rings have unequal numbers of peaks.

13. The stent assembly of claim 12 wherein the ostial ring has more peaks than the outermost ring.

14. The stent assembly of claim 12 in which there are more than two rings and in which the number of peaks on each ring increases progressively from the outermost ring to the ostial ring.

15. The stent assembly of claim 12 wherein the outermost ring has more peaks than the ostial ring.

16. The stent assembly of claim 12 in which there are more than two rings and in which the number of peaks on each ring increases progressively from the ostial ring to the outermost ring.

17. The stent assembly of claim 10 wherein the at least two rings comprise a first ring and a second ring and the number of peaks on the first ring relative to the number of peaks on the second ring is one selected from the list consisting of: 8:16, 8:24, 16:16, and 16:24.

18. A bifurcated stent assembly having an expanded state and an unexpanded state, the assembly comprising:
a substantially tubular, first stent body defining a first lumen with a proximal end and a distal end, the first lumen positioned within a circumferential plane, having a first longitudinal axis extending therethrough and having a side opening along the circumferential plane;
and a second stent body;
the second stent body being connected to the first stent body, positioned adjacent to the side opening, and comprising a plurality of interconnected rings;
in the unexpanded state at least two of the rings being positioned substantially along the circumferential plane of the first stent body each of the at least two rings having a plurality of peaks and spans in between these peaks wherein the product of the number of peaks of one ring multiplied by the length of the span of that ring is greater than the product of number of peaks of a second ring multiplied by the length of the span of the second ring and;
in the expanded state, the interconnected rings of the second stent body are positioned farther away from each other than in the unexpanded state and define a second lumen and the second stent body is in fluid communication with the first lumen.

19. The stent of claim 18 having one ring being an ostial ring positioned closest to the junction of the first and second stent bodies and one ring being an outermost ring positioned farthest away from the junction of the first and second stent bodies wherein the rings form a tapering second lumen having a ring with a greatest width and a ring with a narrowest width in which the rings progressively widen in the direction of the outermost end.

20. The stent of claim 18 having one ring being an ostial ring positioned closest to the junction of the first and second stent bodies and one ring being an outermost ring positioned farthest away from the junction of the first and second stent bodies wherein the rings form a tapering second lumen having a ring with a greatest width and a ring with a narrowest width in which the rings progressively widen in the direction of the ostial end.
